(19) United States
(12) Patent Application Publication
(10) Pub. No.: US 2006/0222700 A1
(43) Pub. Date: Oct. 5, 2006

(54) Tablet and capsule form of liquid active ingredient

(76) Inventor: Pieter J. Groenewoud, Powder Springs, GA (US)

Correspondence Address: BRYAN W. BOCKHOP, ESQ., 2375 MOSSY BRANCH DR., SNELLVILLE, GA, 30078, US

(21) Appl. No.: 11/098,799

(22) Filed: Apr. 5, 2005

(51) Int. Cl. A61K 9/48; A61K 9/20

(52) U.S. Cl. 424/451; 424/464

(57) ABSTRACT

A tablet includes a liquid active ingredient, such as benzonatate, and a powdered material, such as fumed silica. The powdered material has adsorbed a high proportion by weight of the liquid active ingredient while maintaining characteristics of a solid. The liquid active ingredient and the powdered material are compressed to form a tablet. To make the tablet, the liquid active ingredient is mixed with the powdered material in a proportion that retains solid properties. A binder is added to the solid-like substance to form a combination. The combination is granulated to form granules. The granules are compressed to form tablets. The material may also be disposed in capsules.

### BACKGROUND OF THE INVENTION

1. Field of the Invention

The present invention relates to pharmaceuticals and, more specifically, to a tablet form of a liquid active ingredient.

2. Description of the Prior Art

Certain pharmaceutical compositions, such as benzonatate—an antitussive, are manufactured in liquid form. Typically, dosages of benzonatate are placed in a soft gelatin capsule (“gel cap”). Some patients, especially children, have a tendency to bite into soft gel caps, expecting them to taste like candy. When bitten hard enough, the liquid in the gel cap is released into the patient's mouth. Benzonatate causes local anesthesia in the mouth and throat. This local anesthesia can lead to choking in certain patients. Therefore, there is a need for liquid for pharmaceuticals in a form with a reduced likelihood of being released in the patient's mouth.

Pharmaceutical compositions may be delivered in tablet form. Tablets offer the advantages of low cost and stability. Tablets are also unlikely to be bit into by patients and, even if a patient accidentally bites a tablet, only a relatively little amount of the active ingredient will be released due to the biting. Currently, however, there is no method for making a tablet for delivering a liquid active ingredient.

Placing liquid active ingredients in gel caps is an expensive process that requires use of solvents and other expensive machinery. Hard capsules require special sealing machinery if they are to hold liquid ingredients, which adds to their expense.

Therefore, there is a need for a tablet that delivers a liquid active ingredient.

There is also a need for a method of making a tablet from a liquid active ingredient.

There is also a need for a method of inexpensively encapsulating a liquid active ingredient.

### SUMMARY OF THE INVENTION

The disadvantages of the prior art are overcome by the present invention which, in one aspect, is a tablet that includes a liquid active ingredient and a powdered material. The powdered material has adsorbed a high proportion by weight of the liquid active ingredient while maintaining characteristics of a solid. The liquid active ingredient and the powdered material are compressed to form a tablet.

In another aspect, the invention is a benzonatate formulation that includes liquid benzonatate and fumed silica. The liquid benzonatate is mixed with the fumed silica to form a solid-like substance.

In another aspect, the invention is a capsule that includes a liquid active ingredient and a powdered material. The powdered material has adsorbed a high proportion by weight of the liquid active ingredient while maintaining characteristics of a solid. The liquid active ingredient and the powdered material are disposed in a capsule shell.

In yet another aspect, the invention is a method of making a tablet from a liquid active ingredient, in which the liquid active ingredient is mixed with a powdered material capable of adsorbing a high proportion of liquid while retaining solid properties to form a solid-like substance. The liquid active ingredient is in a proportion to the powdered material that retains solid properties. A binder is added to the solid-like substance to form a combination. The combination is granulated to form granules. The granules are compressed to form tablets.

These and other aspects of the invention will become apparent from the following description of the preferred embodiments taken in conjunction with the following drawings. As would be obvious to one skilled in the art, many variations and modifications of the invention may be effected without departing from the spirit and scope of the novel concepts of the disclosure.

### BRIEF DESCRIPTION OF THE FIGURES OF THE DRAWINGS

FIG. 1 is a schematic diagram showing a method for making a tablet or a capsule that holds a liquid active ingredient.

### DETAILED DESCRIPTION OF THE INVENTION

A preferred embodiment of the invention is now described in detail. Referring to the drawings, like numbers indicate like parts throughout the views. As used in the description herein and throughout the claims, the following terms take the meanings explicitly associated herein, unless the context clearly dictates otherwise: the meaning of “a,” “an,” and “the” includes plural reference, the meaning of “in” includes “in” and “on.”

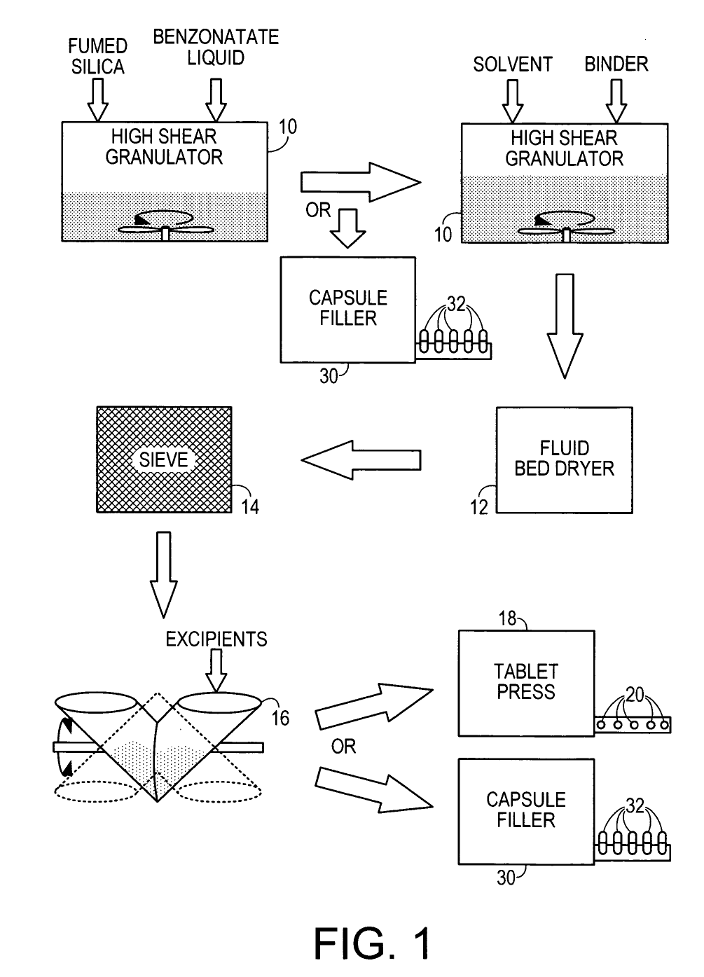

As shown in FIG. 1, one illustrative embodiment of a method of making a tablet according to the invention includes adding a liquid active ingredient, such as benzonatate, to a powder, such as fumed silica, in a high shear granulator 10. Fumed silica is an amorphous, crystalline-free form of silicon dioxide with extremely small particle size, extremely high surface area and high purity. One example of suitable fumed silica is AEROSIL®, another example is AEROPERL® granulated fumed silica; both are available from Degussa Corporation, Aerosil & Silanes, 2 Turner Place, Piscataway, N.J. 08855. The high shear granulator 10 is allowed to run until the liquid active ingredient is substantially completely adsorbed by the fumed silica (e.g. five minutes in one example). At this point, the liquid active ingredient and fumed silica mixture has retained the qualities of a powder. Typically, with the certain fumed silicas, liquid in an amount of up to about 68% of the weight of the fumed silica may be added to the fumed silica with the fumed silica and liquid mixture still retaining the properties of a solid.

A solvent (e.g., water or alcohol) and a binder (e.g., hydroxypropyl methylcellulose) are then added to the liquid active ingredient and fumed silica mixture. The high shear granulator 10 is restarted and allowed to run until the new mixture is thoroughly mixed. The result is a paste-like substance. The paste-like substance is dried in a fluid bed dryer 12 to form granules. The granules are separated by size in a sieve 14 and granules of a desired size range are added to other excipients (such as lubricants and disintegrants) and mixed in a mixer 16. The resulting mixture of granules and excipients is then compressed in a tablet press 18 to form a plurality of tablets 20.

The tablets 20 may be coated with a coating that isolates the tablet from a user's throat tissues when swallowing. The coating is made a material that is soluble in the digestive system. One example of a suitable coating is hydroxypropyl methylcellulose. Many other suitable coatings are well known to the pharmaceutical arts. Typically, the coating is sprayed onto the tablets 20 and dried in a rotary drier (not shown).

In a second illustrative embodiment, the combination of the liquid active ingredient and the fumed silica can be disposed in a capsule 32 using a conventional pharmaceutical capsule filler 30. This embodiment stabilizes the liquid active ingredient by adsorbing it to the fumed silica and allows it to be used to fill a capsule. This is a relatively inexpensive method of delivering the active ingredient. Alternately, the fumed silica/liquid active ingredient may be granulated, as described above, and then disposed in the capsules 32. The granules may be sprayed with a non-immediate release (e.g., timed release or delayed release) coating prior to placement in the capsules 32.

The above described embodiments are given as illustrative examples only. It will be readily appreciated that many deviations may be made from the specific embodiments disclosed in this specification without departing from the invention. Accordingly, the scope of the invention is to be determined by the claims below rather than being limited to the specifically described embodiments above.

What is claimed is:

1. A tablet, comprising
a. a liquid active ingredient; and
b. a powdered material that has adsorbed a high proportion by weight of the liquid active ingredient while maintaining characteristics of a solid, the liquid active ingredient and the powdered material compressed to form a tablet.

2. The tablet of claim 1, further comprising a coating about the tablet that isolates the tablet from a user's throat tissues when swallowing, the coating being made of a material that is soluble in the user's digestive system.

3. The tablet of claim 1, wherein the powdered material comprises fumed silica.

4. The tablet of claim 1, wherein the liquid active ingredient comprises benzonatate.

5. The tablet of claim 1, further comprising at least one excipient.

6. The tablet of claim 5, wherein the excipient comprises a binder.

7. The tablet of claim 5, wherein the excipient comprises a disintegrant.

8. The tablet of claim 5, wherein the excipient comprises a lubricant.

9. A capsule, comprising
a. a liquid active ingredient;
b. a powdered material that has adsorbed a high proportion by weight of the liquid active ingredient while maintaining characteristics of a solid; and
c. a capsule shell into which the liquid active ingredient and the powdered material are disposed.

10. The capsule of claim 9, wherein the powdered material comprises fumed silica.

11. The capsule of claim 9, wherein the liquid active ingredient comprises benzonatate.

12. The capsule of claim 9, further comprising at least one excipient.

13. The capsule of claim 9, wherein the liquid active ingredient and the powdered material are formed into granules.

14. The capsule of claim 13, wherein the granules are coated with a non-immediate release coating.

15. A benzonatate formulation, comprising:
a. fumed silica; and
b. liquid benzonatate mixed with the fumed silica to form a solid-like substance.

16. The benzonatate formulation of claim 15, wherein the solid-like substance is compressed into tablet form.

17. The benzonatate formulation of claim 15, wherein the solid-like substance is encapsulated in a capsule.

18. A method of making a tablet from a liquid active ingredient, comprising the steps of:
a. mixing the liquid active ingredient with a powdered material capable of adsorbing a high proportion of liquid while retaining solid properties to form a solid-like substance, the liquid active ingredient in a proportion to the powdered material that retains solid properties;
b. adding a binder to the solid-like substance to form a combination;
c. granulating the combination to form granules; and
d. compressing the granules to form tablets.

19. The method of claim 18, wherein the liquid active ingredient comprises benzonatate.

20. The method of claim 18, further comprising the step of adding a binder to the solid-like substance.

21. The method of claim 18, further comprising the step of adding a lubricant to the solid-like substance.

22. The method of claim 18, further comprising the step of adding a disintegrant to the solid-like substance.

23. The method of claim 18, further comprising the step of adding a solvent to the combination prior to the granulation step.

24. The method of claim 18, further comprising the step of sieving the granules to separate granules of a similar size.

25. The method of claim 18, further comprising the step of coating the tablets with a coating that isolates the tablet from a user's throat tissues when swallowing, the coating being made of a material that is soluble in the user's digestive system.

26. The method of claim 25, wherein the coating step comprises spraying the coating onto the tablets.
